(19) United States
(12) Patent Application Publication
(10) Pub. No.: US 2023/0149102 A1
(43) Pub. Date: May 18, 2023

(54) INTERVENTIONAL SURGICAL ROBOT SYSTEM, CONTROL METHOD AND MEDIUM

(71) Applicant: BEIJING WEMED MEDICAL EQUIPMENT CO.,LTD., Beijing (CN)

(72) Inventors: Tao HUANG, Beijing (CN); Lin WANG, Beijing (CN)

(73) Assignee: BEIJING WEMED MEDICAL EQUIPMENT CO.,LTD., Beijing (CN)

(21) Appl. No.: 18/089,821

(22) Filed: Dec. 28, 2022

Related U.S. Application Data
(63) Continuation of application No. PCT/CN2022/121200, filed on Sep. 26, 2022.

(51) Int. Cl. A61B 34/32; A61B 34/37; A61B 34/10; A61B 34/20; B25J 9/16; G06T 7/20; G06T 7/00; G06T 7/70; G06T 7/60

(52) U.S. Cl. CPC A61B 34/32; A61B 34/37; A61B 34/10; A61B 34/20; B25J 9/1697; G06T 7/20; G06T 7/0012; G06T 7/70; G06T 7/60; A61B 2034/303

(57) ABSTRACT

An interventional surgical robotic system, control method, and medium are provided. The system includes a master-end mechanism and a slave-end mechanism. The master-end mechanism includes a processor, a display and a user control. The processor acquires an intra-operative image containing a physiological tubular structure, and generate an automatic navigation instruction by performing analysis processing on the intra-operative image. The user control receives manual manipulation of a user and transmit a manual control instruction corresponding to the manual manipulation. The slave mechanism receives instructions from the processor and the user control, and to steer the medical interventional device to advance based on the automatic navigation instruction in case the automatic navigation instruction is received without receiving the manual control instruction, and to steer the medical interventional device based on the manual control instruction in case the manual control instruction is received.

### CROSS-REFERENCE TO RELATED APPLICATIONS

This patent application is a continuation application of PCT/CN2022/121200 filed on Sep. 26, 2022, which claims priority to Chinese patent disclosure No. 202210859807.5, filed on Jul. 22, 2022, the entire contents of which are incorporated herein by reference.

### TECHNICAL FIELD

The disclosure relates to the technical field of interventional surgical robot control, and more specifically, to an interventional surgical robot system, a control method and a medium.

### BACKGROUND

The minimally invasive interventional therapy for the cardiovascular and cerebrovascular diseases is a main treatment means for the cardiovascular and cerebrovascular diseases, and has the obvious advantages of small incision, short postoperative recovery time and the like compared with the traditional surgical operation. The cardiovascular and cerebrovascular interventional operation is a process in which a doctor manually sends a catheter, a guide wire, a stent and other instruments into a patient to finish treatment. The interventional operation has many defects, for example, during the operation, the DSA emits X-rays, the physical strength of a doctor is reduced rapidly, the attention and the stability are also reduced, the operation precision is reduced, and accidents such as endangium injury, vascular perforation and rupture and the like caused by improper pushing force are easy to happen, so that the life risk of a patient is caused. Second, the cumulative damage of long-term ionizing radiation can greatly increase the probability of doctors suffering from leukemia, cancer and acute cataract. The phenomenon that doctors accumulate rays continuously because of interventional operation becomes a problem that the occupational lives of the doctors are damaged and the development of the interventional operation is restricted to be neglected.

The defects can be effectively solved by means of the robot technologies, the precision and the stability of the operation can be greatly improved, meanwhile, the injury of the radioactive rays to the interventional doctor can be effectively reduced, and the occurrence probability of accidents in the operation is reduced. However, the current interventional robots are operated manually, the vascular interventional surgical robot takes a long time to perform the surgical operation, and a doctor needs to concentrate for a long time to perform the surgical operation, which is prone to fatigue and causes misoperation. Therefore, the automatic operation of the cardiovascular and cerebrovascular interventional operation auxiliary robot is more and more concerned by people, and gradually becomes a key research and development object in the field of medical robots in all the science and technology strong countries.

However, a mature and available automatic intervention operation control method for an automatic operation system of a vascular intervention robot is not available, automatic operation cannot be performed, and manual control is usually adopted, so that intervention operation is completed for a long time, the accuracy is not high, and the efficiency is low, thus there are chances for the improvements to be made.

### SUMMARY

The disclosure aims to solve the above technical problems in the prior art. The disclosure provides an interventional surgical robot system, a control method and a medium, which can realize an automatic navigation function and interactions between the interventional surgical robot system and a doctor. The interventional surgical robot can be guided, by the doctor, to operate automatically and the doctor supervises and solves problems encountered in the automatic operation process in time so as to improve the accuracy and the safety of the automatic operation of the interventional surgical robot.

According to a first aspect of the disclosure, an interventional surgical robotic system for manipulating a medical interventional device for movement within a lumen of a physiological tubular structure of a patient is provided. The interventional surgical robot system includes a master-end mechanism and a slave-end mechanism. The master-end mechanism includes at least one processor, a display and a user control. The at least one processor is configured to acquire an intra-operative image containing the physiological tubular structure, and to generate the automatic navigation instruction by performing an analysis process on the intra-operative image. The display is used for displaying the intra-operative image and the current motion state of the medical interventional device. The user control is configured to receive manual manipulation of a user and transmit a manual control instruction corresponding to the manual manipulation. The slave-end mechanism is provided with a mechanical arm and an end actuator, and is configured to receive instructions from the at least one processor and the user control, to steer a medical interventional device to advance based on the automatic navigation instructions in the case where the automatic navigation instructions are received without receiving the manual control instructions, and to steer the medical interventional device based on the manual control instructions in the case where the manual control instructions are received.

According to a second aspect of the disclosure, a control method of an interventional surgical robot for manipulating a medical interventional device for movement within a lumen of a physiological tubular structure of a patient is provided. An intra-operative image containing the physiological tubular structure via at least one processor of a main-end mechanism is acquired, and an automatic navigation instruction by performing analysis processing on the intra-operative image is generated. The intra-operative image and a current motion state of the medical interventional device are presented via a display. Manual operation of a user through a user control is received, and a manual control instruction corresponding to the manual operation is transmitted. Instructions from the at least one processor and the user control via a slave-end mechanism are received. The slave-end mechanism is provided with a mechanical arm and an end actuator. The slave-end mechanism, upon receiving an automatic navigation instruction without receiving the manual control instruction, manipulates a medical interventional device to advance based on the automatic navigation instruction, and upon receiving the manual control instruction, manipulates a medical interventional device based on the manual control instruction.

According to a third aspect of the disclosure, a computer-readable storage medium having stored thereon computer program instructions which, when executed by a processor, cause the processor to execute a control method of an interventional surgical robot according to various embodiments of the disclosure is provided.

Compared with the prior art, the beneficial effects of the embodiment are as follows.

The interventional surgical robot system provided by the embodiment of the disclosure can automatically complete main operation steps in an operation when a blood vessel interventional operation is carried out. The master-end mechanism of the interventional surgical robot system comprises a processor, the processor acquires an intra-operative image containing a physiological tubular structure, and an automatic navigation instruction can be generated by identifying the image and analyzing the intra-operative image. In the real-time surgical procedure, the execution state of the end actuator of the interventional surgical robot is also changed along with the execution of the automatic surgical operation of the interventional surgical robot. The interventional surgical robot system provided by the embodiment of the disclosure can generate an automatic navigation instruction, and the automatic navigation instruction, can guide and control the slave-end mechanism of the robot to complete the control action on the end actuator in real time so as to enable the end actuator to reach the designated position. During automatic surgical execution, the interventional surgical robotic system gives the physician intra-operative real-time parameter information. The doctor can adjust the interventional surgical robot system by checking the relevant parameters fed back by the system through human-computer interaction with the interventional surgical robot system so as to improve the accuracy of the generated automatic navigation instruction, and can control the slave mechanism to pause at any time in the automatic operation process and check and correct the execution condition of the slave mechanism. So, can effectively realize doctor and intervene the human-computer interaction of operation robot system, intervene operation robot system and not only can realize automatic operation, moreover, through the human-computer interaction of this kind of doctor and system, realize the multiple safety protection to automatic operation execution process, can effectively protect the safety of automatic execution operation in-process.

The above description, is only an overview of the technical solutions of the disclosure, and the disclosure may be implemented in accordance with the content of the description so as to make the technical means of the disclosure more clearly understood, and the detailed description of the disclosure will be given below in order to make the above and other objects, features, and advantages of the disclosure more clearly understood.

### BRIEF DESCRIPTION OF DRAWINGS

In the drawings, which are not necessarily drawn to scale, same reference numerals may indicate similar components in different views. Identical reference numerals having letter suffixes or different letter suffixes may represent different examples of similar components. The drawings illustrate generally, by way of example, but not by way of limitation, various embodiments and, together with the description and the claims, serve to explain the disclosed embodiments. Such embodiments are illustrative and exemplary and are not intended to be exhaustive or exclusive embodiments of the present method, apparatus, system, or non-transitory computer-readable medium having instructions for implementing the method.

FIG. 1(a) is a schematic diagram showing the composition of an interventional surgical robotic system according to an embodiment of the disclosure.

FIG. 1(b) is a schematic diagram of an overall structual of an interventional surgical robotic system according to an embodiment of the disclosure.

FIG. 1(c) is a flowchart of an overall method for performing an automated surgery according to an interventional surgical robotic system according to an embodiment of the disclosure.

FIG. 2 is a flowchart of a method for generating automatic navigation instructions for an interventional surgical robotic system according to an embodiment of the disclosure.

FIG. 3 is a schematic diagram of an interventional surgical robotic system generating automatic navigation instructions according to an embodiment of the disclosure.

FIG. 4 is a flow chart of a method of generating an automated navigation instruction to reduce an advancing speed for an interventional surgical robotic system according to an embodiment of the disclosure.

FIG. 5 is a flow chart of a method of controlling an automated surgical procedure based on a deviation by an interventional surgical robotic system according to an embodiment of the disclosure.

FIG. 6 is a flowchart of a method for human-computer interaction during automatic surgery of an interventional surgical robotic system according to an embodiment of the disclosure.

FIG. 7 is a flowchart of a control method of an interventional surgical robot according to an embodiment of the disclosure.

### DETAILED DESCRIPTION OF THE EMBODIMENTS

In order to make the technical solutions of the disclosure better understood, the disclosure is described in detail below with reference to the accompanying drawings and the detailed description. The embodiments of the disclosure will be described in further detail below with reference to the drawings and specific embodiments, but the disclosure is not limited thereto.

As used in this disclosure, the terms “first”, “second” and the like, do not denote any order, quantity, or importance, but are rather are used to distinguish one element from another. The use of the word “comprising” or “comprises” and the like in this disclosure is intended to mean that the elements listed before this word cover the elements listed after this word and not to exclude the possibility that other elements may also be covered. In the disclosure, arrows shown in the drawings of the respective steps are only used as examples of execution sequences, and are not limited, and the technical solution of the disclosure is not limited to the execution sequences described in the embodiments, and the respective steps in the execution sequences may be executed in a combined manner, may be executed in a split manner, and may be in an order-changed manner as long as the logical relationship of the execution content is not affected.

All terms (including technical and scientific terms) used herein have the same meaning as commonly understood by one of ordinary skill in the art to which this disclosure belongs unless specifically defined otherwise. It will be further understood that terms, such as those defined in commonly used dictionaries, should be interpreted as having a meaning that is consistent with their meaning in the context of the relevant art and will not be interpreted in an idealized or overly formal sense unless expressly so defined herein. Techniques, methods, and apparatus known to one of ordinary skill in the relevant art may not be discussed in detail but are intended to be part of the specification where appropriate.

An interventional surgical robotic system is provided according to embodiments of the disclosure and may include, for example, a master-end mechanism, a slave-end mechanism. The master-end mechanism and the slave-end mechanism respectively execute corresponding steps in the control method of the interventional surgical robot according to various embodiments of the disclosure.

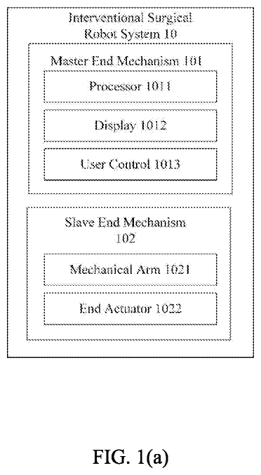

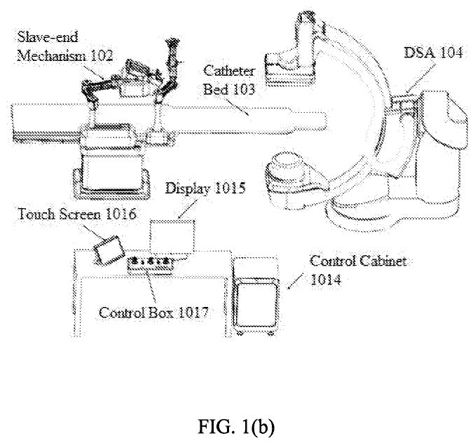

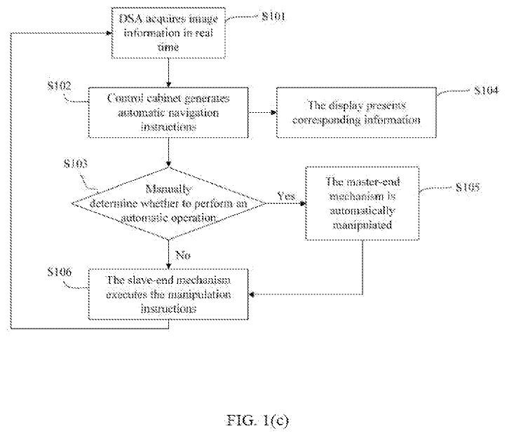

FIG. 1(a) shows a schematic composition diagram of an interventional surgical robotic system according to an embodiment of the disclosure. The interventional surgical robotic system 10 is used to manipulate a medical interventional device for movement within a lumen of a physiological tubular structure of a patient. The interventional surgical robotic system 10 includes a master-end mechanism 101 and a slave-end mechanism 102. The master-end mechanism 101 includes at least one processor 1011, a display 1012, and a user control 1013.

In this embodiment, the at least one processor 1011 is configured to acquire an intra-operative image containing the physiological tubular structure, and to generate the automatic navigation instruction by performing an analysis on the intra-operative image. The image may be a blood vessel image acquired from an image database or an image acquired based on other methods, and is not limited in particular. The acquisition modality for the image includes, but is not limited to, direct acquisition by various imaging modalities, such as, but not limited to, intra-operative contrast imaging techniques such as DSA, endoscopy, etc., or post-processing or reconstruction based on the raw image acquired by the imaging device. The technical term “acquisition” refers herein to any manner of direct or indirect acquisition, with or without additional noise reduction, cropping, reconstruction, etc. image processing.

The intra-operative is understood to be during surgery, rather than pre-operatively and post-operatively. For example, the following description will be given by taking an example of advancing a guide wire through a blood vessel. In the operation, the advancing position, the advancing distance, the changing angle of the guide wire head, the curvature of the blood vessel, the stenosis degree and other relevant motion parameters of the guide wire are changed along with the automatic operation, the change greatly improves the operation difficulty of the automatic operation, and an interventional surgical robot for the automatic operation has difficulty in obtaining a safe and correct advancing, path and operation mode and has greater difficulty compared with the prediction of the advancing path and the operation mode before and after the operation (the relevant parameters are relatively stable under the states of the operation and the operation). The processor 1011 generates an automatic navigation instruction for guiding the medical interventional device to move in the cavity of the physiological tubular structure by analyzing and processing the image in the operation, and performs automatic operation based on the automatic navigation instruction, so that the efficiency of the automatic operation is improved.

In particular, the processor 1011 may be a processing device such as a microprocessor, central Processing Unit (CPU), graphics Processing Unit (GPU), etc., which may include one or more general purpose processing devices. More specifically, the processor 1011 may be a Complex Instruction Set Computing (CISC) microprocessor, reduced Instruction Set Computing (RISC) microprocessor, very Long Instruction Word (VLIW) microprocessor, processor executing other instruction sets, or processors executing a combination of instruction sets. Processor 1011 may also be one or more special-purpose processing devices such as an disclosure Specific Integrated Circuit (ASIC), a Field Programmable Gate Array (FPGA), a Digital Signal Processor (DSP), a system on a chip (SoC), or the like. As will be appreciated by those skilled in the art, in some embodiments, the processor 1011 may be a dedicated processor rather than a general purpose processor. Processor 1011 may include one or more known processing devices such as a microprocessor from the Pentium™, core™, xeon™ or Itanium™ family manufactured by Intel™, turion™, athlon™, sempron™, opteron™, FX™, phenom™ family manufactured by AMD™, or various processors manufactured by Sun. Microsystems. The processors 1011 may also comprise graphics processing units, such as GPU from GeForce®, quadro® produced by Nvidia, tesla series, GMA produced by Intel™, iris™ series, or Radeon™ series produced by AMD™. Processor 1011 may also include an accelerated processing unit such as the Desktop A-4 (6, 6) family manufactured by AMD™, the Xeon Phi™ family manufactured by Intel™. The disclosed embodiments are not limited to any type of processor or processor circuit that is otherwise configured to perform a method of controlling an interventional surgical robot in accordance with various embodiments of the disclosure. In addition, the term “processor” or “image processor” may include more than one processor, e.g., a multi-core design or multiple processors, each having a multi-core design. The processor 1011 may execute sequences of computer program instructions stored in the memory to perform the various operations, processes and methods disclosed herein. The processor 1011 may be communicatively coupled to a memory and configured to execute computer-executable instructions stored therein.

The display 1012 is used for displaying the intra-operative image and the current motion state of the medical interventional device, so that a doctor or other users aware the current motion state of the medical interventional device in time. For example, whether the display result is as expected or not can be judged through the intra-operative image presented by the display 1012, or whether the motion state such as the position of the medical access device in the blood vessel is in a safe and correct range or not can be judged in time, so that when a safety problem is about to occur, manual intervention can be performed on the automatic surgical process of the interventional surgical robot in advance. The display unit 1012 may be, for example, the display 1015 shown in FIG. 1(b), or may be a member in which the display 1015 is engaged with another device, which is not particularly limited.

The user manipulating part 1013 is configured to receive a manual manipulation by a user and transmit a manual control instruction corresponding to the manual manipulation, so that the user can effectively control a process of performing an automatic operation by the interventional surgical robot. As shown in FIG. 1(b), the user control 1013 includes, but is not limited to, a control box 1017, and the control box 1017 is used for a doctor to perform an operation of manually controlling the robot. Taking the example of using the robot to advance and rotate the guide wire and the guide tube in the blood vessel, when the robot acts wrongly or has other emergencies, the doctor can control the robot to complete the operation of the catheter and the guide wire by controlling the rocker and the roller on the control box 1017, thereby ensuring the smooth operation. The control box 1017 may include two ways to transmit manual control commands to the slave-end mechanism 102. For example, a circuit board may be embedded in the control box 1017, and manual control commands are sent directly to the slave mechanism 102 via the control box 1017. Alternatively, the control box 1017 transmits the manual control command to the processor 1011, which may be subsequently forwarded to the slave-end mechanism 102 via a relay device in the master-end mechanism 101, such as, but not limited to, the control cabinet 1014 in FIG. 1(b).

Further, the slave-end mechanism 102 is provided with a robotic arm 1021 and an end actuator 1022. For example, the end actuator 1022 is a guide wire actuator and/or a catheter actuator, and cooperates with DSA 104 (as shown in FIG. 1(b)) to perform an operation action for the interventional procedure. The guide wire actuator is used for clamping the guide wire to push and rotate the guide wire under the action of the mechanical arm 1021, and the catheter actuator is used for clamping the catheter to push and rotate the catheter under the action of the mechanical arm 1021. As shown in FIG. 1(b), the slave-end mechanism 102 is mounted on the catheter bed 103, the DSA104, catheter bed 103 and interventional surgical robot are placed in the catheter chamber, the master-end mechanism 101 is placed in the control chamber, the master-end mechanism 101 includes, but is not limited to, a control cabinet 1014, a display 1015, a touch screen 1016 and a control cabinet 1017, and the control cabinet 1017. The touch screen 1016 and the display 1015 are all connected to the control cabinet 1014. The physician can view the conditions within the catheter chamber through the lead glass window of the catheter chamber in the control room. Control cabinet 1014 includes at least one processor 1011 that may acquire and analyze image information acquired from DSA104, generate and send autopilot commands to slave-side mechanism 102, and slave-side mechanism 102 may also feed back information related to the data to perform actions to control cabinet 1014. The control cabinet 1014, includes but not limited to, a processor 1011 for data analysis processing, a UPS for power supply, an isolation transformer for voltage stabilization and switching power supply lamps. The display 1015 presents information including, but not limited to, automated navigation instructions, motion state image information of the medical interventional device, and anticipated actions of the robot, path planning, and the like. The touch Screen 1016 is used for human-computer interaction, such as parameter setting, command confirmation, etc., and also presents real-time resistance information of the robot detection catheter and guide wire.

The slave mechanism 102 is configured to receive instructions from the at least one processor 1011 and the user control 1013. When an automatic navigation instruction is received without receiving the manual control instruction, the slave mechanism operates the medical interventional device to advance based on the automatic navigation instruction, and when the manual control instruction is received, the slave mechanism operates the medical interventional device based on the manual control instruction, so that the human-computer interaction between the doctor and the interventional surgical robot, which is compatible with workload and safety performance is realized. The slave mechanism can automatically navigate without the manual control instruction. The doctor only needs to monitor in real time, and when the doctor finds any problem, the slave mechanism can send the manual control instruction at any time to operate the automatic navigation instruction to rapidly intervene, and the safety and the accuracy of the automatic operation are improved.

Specifically; as shown in FIG. 1(c), in step S101, DSA104 acquires image information in real time, and in master-end mechanism 101, processor 1011 in control cabinet 1014 can generate automatic navigation instructions by analyzing the images during operation acquired by DSA104 in real time (step S102), and present relevant information on display 1015 (step S104), including but not limited to automatic navigation instructions, images during operation, current motion state of medical interventional devices (e.g. guide wires, catheters), next action instructions, planning paths, etc., without specific limitation. Based on the relevant information presented by the display 1015, the physician may intervene manually and make corresponding adjustments. For example, in the automatic operation process, the interventional surgical robot feeds back the real-time image during the operation and the current motion state of the medical interventional device to the display 1015, so that the doctor is informed of the state and the motion of the robot in real time and can perform manual intervention at any time when the doctor does not deal with the situation. For example, the physician can modify relevant parameters in the automated navigation instructions or other relevant parameters on the touch screen 1016 to improve the accuracy and safety of the automated navigation instructions. For another example, in step S103, the doctor may manually determine whether to perform an automatic operation based on the related information. When performing the automatic surgery, the robot performs the automatic surgery based on the automatic navigation instruction, and the master-end mechanism 101 is automatically manipulated (step S105) to perform the automatic surgery. When the doctor thinks there is a high risk in continuing to perform the automatic operation, manual intervention is needed, the doctor enters the manual intervention step by clicking the button on the touch screen 1016, and the robot system temporarily stops the automatic operation and waits for the doctor to make an adjustment. For example, the doctor can manually operate the robot by controlling the two devices, i.e., the touch screen 1016 and the control box 1017, and can also send instructions to the slave-end mechanism 102 of the robot, and based on the manual control instructions, the slave-end mechanism 102 executes the manipulation instructions (step S106). After the completion of the doctor's adjustment, the automatic operation mode can continue to resume, and the steps in sequence will be cycled until the task is completed. The strategy can realize real-time, accurate and safe automatic operation on the patient under the condition of manual supervision, which greatly improves the operation experience of doctors, reduces the physical burden of the doctors, and has higher practicability and research value in the field of medical robots. Under the condition that the slave-end mechanism 102 does not receive the manual control instruction, the vessel intervention operation robot controls the slave-end mechanism 102 to move according to the automatic navigation instruction so as to drive the catheter and/or the guide wire to move, then the motion information is fed back to the control cabinet 1014, the DSA104 image is changed after the guide wire and/or the catheter move. The automatic navigation instruction is timely updated based on the updated intra-operative image, and the execution of the automatic operation is promoted.

Thus, through efficient human-computer interaction between the doctor and the interventional surgical robot system 10, the execution efficiency of automatic surgery performed by the interventional surgical robot is improved, and the accuracy and the safety of the surgery are greatly improved.

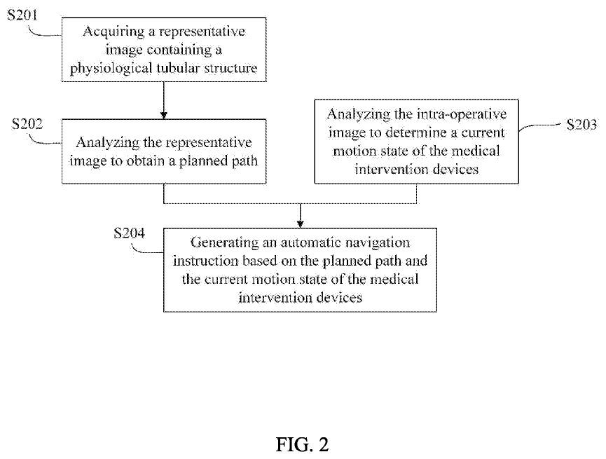

FIG. 2 shows a flowchart of a method of generating automatic navigation instructions for the interventional surgical robotic system 10 according to an embodiment of the disclosure. In step S201, a representative image containing a physiological tubular structure is acquired, and the image data, sources include at least medical image information acquired from the DSA104 and a large amount of doctor's clinical operation data. The acquired medical image information refers to an image acquired by DSA104 digital subtraction angiography, and the specific part of the acquired data is not limited, including but not limited to nerves, the thoracic cavity, and the like. The doctor operation data refers to operation data performed by a doctor through a screen of an interventional robotic automatic surgery system (hereinafter referred to as a robotic system) or data in a conventional clinical operation. In step S202, the representative image is analyzed to obtain a planning path, for example, a learning network may be used to extract a blood vessel centerline from the representative image, and the extracted blood vessel centerline is used as the planning path, and a method for specifically generating the planning path will be described in detail below. The planning path described herein may be understood as a path along which the medical interventional device moves along a true extending direction of the blood vessel. The planning path can be acquired through system calculation, can also be manually set by a doctor, or can be acquired through manual correction based on the calculation result of the system. In step S203, the intra-operative image is analyzed to determine a current motion state of the medical interventional device, which may be understood as a current motion trend of the medical interventional device, such as a forward movement, a rotation or other motion trend of the medical interventional device. The current motion state may also include a direction of motion, an angle, etc. of the medical interventional device at the current time. The current motion state is not particularly limited, specifically subject to the requirements in the actual operation process. In step S204, an automatic navigation instruction is generated based on the planning path and the current motion state of the medical interventional device to improve the safety of the interventional surgical robot in performing the automatic surgery.

In some embodiments, the step of generating the automatic navigation instruction based on the planning path and the current motion state of the medical interventional device specifically includes obtaining a current first position and a first, motion direction of a representative portion of the medical interventional device, determining a second position of the representative portion in the first motion direction, determining a shortest connecting line between the second position of the representative portion and the planning path, determining an intersection point of the shortest connecting line and the planning path, obtaining an included angle between a connecting line of the intersection point and the first position and the first motion direction, and when the included angle is smaller than a first threshold angle, generating an advancing automatic navigation instruction so that the medical interventional device moves forward upon receiving the advancing automatic navigation instruction. The representative part includes, but is not limited to, the medical interventional device itself, the head of the medical interventional device, or other parts designated by the user and capable of calibrating the motion change of the medical interventional device. The representative portion is not particularly limited, but depends on the actual condition of the doctor in the actual operation process.

Wherein the first position may be understood as the position of the medical interventional device before the next action is performed. For example, if the medical interventional device moves forward from the a position to the b position and then moves forward from the b position to the c position, the a position is the first position relative to the b position, and the b position is the first position relative to the c position. The first direction of movement is similar to the first position, which may be understood as the direction of movement of the medical interventional device before the next action is performed, and the determination of the direction of movement may be manually set by the physician based on the actual direction of movement in which the physician advances the medical interventional device. Alternatively, the direction may be a tangential direction at the corresponding position, or may be a direction set by a computer, which is not particularly limited.

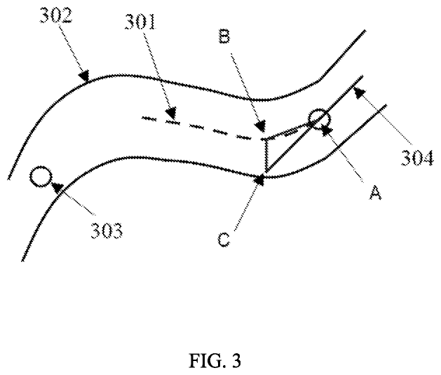

Specifically, the advancing and rotation of the guide wire in the blood vessel are taken as examples. As shown in FIG. 3, the 301 is the planning path, 302 is the blood vessel, 303 is the terminal point, and 304 is the guide wire. The representative part of the guide wire 304 is a guide wire head, and when the guide wire head is at the initial position, the guide wire head is located at a first position a on the planning path 301, and a first moving direction at the first position a is an extending direction of the AC. Then, a second position C is determined in the first motion direction, and the second position C is connected with the planning path 301 to obtain a shortest connecting line BC, wherein a point B is an intersection point of the shortest connecting line BC and the planning path 301. The determination method of the second position C is not specifically limited, and for example, the determination method may be one position arbitrarily selected by the processor 1011 in the first motion direction, as long as the included angle ∠BAC acquired based on the second position is not greater than the first threshold angle. As another example, by manually setting or system setting a default guidewire advancing speed, assuming a 2 mm/s advance speed for the guidewire 304, the processor 1011 in the control cabinet 1014 may determine the second position in the first direction of motion at a advance distance of 0.5s apart. For example, the processor 1011 calculates in advance to determine the second position at 0.5s of advance, and calculates whether the included angle ∠BAC is smaller than a first threshold angle, and if so, generates a forward automatic navigation instruction. At this point, the guidewire 304 can continue to advance upon receiving the navigation instruction to advance, and update the second position to the first position, continuing the above-described process. The speed at which the guidewire 304 is advanced during advancing of the guidewire 304 can be adjusted as needed at any time. The first threshold angle may be an angle manually set by a user, or may be an angle acquired by other methods, which is not limited to this. The above is only one embodiment and does not exclude other methods of determining the second position.

In some embodiments, the distance between the second position and the first position is determined to be the advancing distance when the included angle does not exceed a first threshold angle. Continuing with FIG. 3 as an example, assuming that ∠BAC equal to the first threshold angle, the distance between the first position a and the second position C is the advance distance. Under automated navigation instructions based on this advancing distance, the user may know that it is safe to advance the guidewire 304 from the first position a to the second position C, and that it is nearly consistent with the planning path 301. If the guidewire 304 continues to advance past the second location C, a hazard may arise and the system may issue an alert prompting the user for verification. When receiving the automatic navigation instruction of the advance distance from the end mechanism 102, the guide wire 304 may be directly controlled to advance to the second position C according to the advance distance, or the guide wire 304 may be directly rotated and then advanced, in which the specific embodiment mode is based on the setting of the system and manual operation by the user. In other embodiments, when ∠BAC is less than the first threshold angle, the guide wire 304 is advanced according to a safe advance path that matches the planning path 301, and the processor 1011 may automatically generate an automatic navigation command for advancing according to the setting, or may automatically generate an automatic navigation command for an advancing distance according to the setting, so as to indicate the distance that the guide wire 304 advances forward.

In some embodiments, when the included angle is greater than a first threshold angle, an angle by which the medical interventional device is to be manipulated to rotate is determined, and an automatic rotation instruction indicating the rotation of the angle is generated as the automatic navigation instruction. The rotation angle does not exceed the first threshold angle, and may be other angles smaller than the first threshold angle, or may be the first threshold angle, and the representative part of the medical interventional device is caused to, fall on or near the planning path by manipulating the medical interventional device to rotate by a certain angle. For example, assuming ∠BAC in FIG. 3 is greater than the first threshold angle, continued advancing of the guidewire 304 may create a hazard, leading to vascular rupture. At this point, an auto-navigation instruction may be generated that instructs the guidewire 304 to rotate. The angle of rotation may be the first threshold angle or may be other reasonable angles less than the first threshold angle. For example, after the guidewire 304 is rotated by a first threshold angle, the head of the guidewire 304 falls at point B on the planning path 301. Alternatively, the guidewire 304 is rotated by an angle less than the first threshold angle, and the head of the guidewire 304 falls near point B. The above examples are merely illustrative, and do not specifically limit the scope of protection.

In addition, various approaches may be included during advancing of the guidewire. For example, when the included angle is less than a first threshold angle, the guidewire may be steered to advance in a first direction of motion while updating the first position. And then, continuously determining a new second position based on the updated first position, recalculating the included angle, and when the included angle is equal to the first threshold angle, directly advancing the head of the guide wire to the second position indicated by the automatic navigation instruction of the advancing distance by the user, or directly rotating the guide wire by the first threshold angle and then continuously advancing. The forward automatic navigation instruction, the forward distance automatic navigation instruction and the rotation automatic navigation instruction are not mutually isolated, but are separately carried out and cooperatively matched. In the automatic operation process, the first position is continuously updated based on the motion state of a medical interventional device such as a guide wire, and the calculation is repeated until a series of automatic navigation instructions are acquired. The control of the advancing distance, the advancing distance and the rotating angle of the medical interventional device is separately carried out and mutually cooperated, and the medical interventional device can execute the automatic operation in a more accurate and safe mode through the cooperative control.

In some embodiments, the planning path remains stable during surgery, such that generation of automated navigation instructions is acquired based on the planning path, thereby enabling improved safety of robotic surgery. The image of the physiological tubular structure comprises a vessel image of at least one of a neurovessel, a visceral vessel and a peripheral vessel, so that a planning path acquired based on the image of the physiological tubular structure is kept stable in operation so as to be distinguished from other vessel images which change at any time in operation. For example, a blood vessel near the aorta is accompanied by the motion of the heart, and its blood vessel image during the operation changes at any time, so that a relatively stable blood vessel image cannot be acquired, and a stable planning path that can be used for generating an automatic navigation command cannot be acquired.

In some embodiments, the manual control instructions include at least one of automatic pause instructions, automatic resume instructions, planning path revision instructions, and manual navigation instructions. For example, when the motion state of the medical interventional device is beyond expectation, the doctor may trigger an auto-pause on the touch screen 1016 (FIG. 1(b)) to generate an auto-pause instruction and send the auto-pause instruction to the slave mechanism 102. After the slave-end mechanism 102 is suspended, the doctor can check that no error exists and can trigger an auto-resume on the touch screen 1016 to generate and send an auto-resume instruction to the slave-end mechanism 102. Secondly, after the planning path is acquired based on the image containing the physiological tubular structure, a doctor can also check the planning path, and if the planning path has a larger deviation from the actual blood vessel distribution condition, the doctor can correct the planning path, so that a planning path correction instruction is generated, and the accuracy of the automatic navigation instruction is further improved. In the robotic surgery, once the doctor finds a problem and considers that the robot cannot continue to perform the robotic surgery, the doctor sends a manual navigation command to the slave mechanism 102 to perform manual manipulation. The display 1012 is further configured to display a planning path, and the displayed planning path is manually changed in response to the planning path modification instruction, so that the user can verify the planning path to ensure that the planning path conforms to the actual condition of the blood vessel.

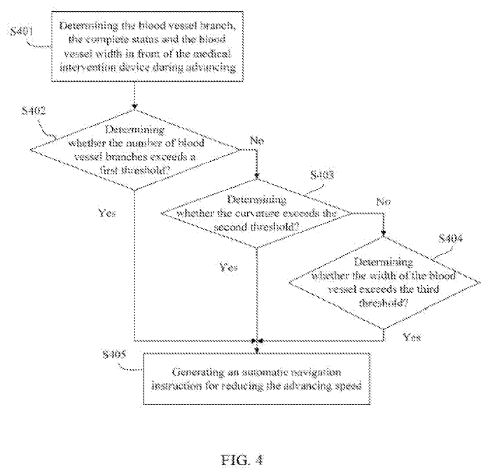

In some embodiments, the intra-operative image is analyzed to determine the vessel branch and curve condition and the vessel width in front of the advancing of the medical interventional device, and if the number of the vessel branches in front exceeds a first threshold, or the curvature is greater than a second threshold. or the vessel width is less than a third threshold, then an automatic navigation instruction for reducing the advancing speed of the medical interventional, device is generated, thereby improving the safety of the automatic operation. Specifically, as shown in FIG. 4, in step S401, the blood vessel branch, the complete status and the blood vessel width before the medical interventional device advances are determined, in step S402, it is determined whether the number of blood vessel branches exceeds a first threshold, if the number of blood vessel branches exceeds the first threshold, it indicates that the blood vessel distribution at the position is complex, which results in a great difficulty in the advancing process of the medical interventional device, at this time, step S405 is executed to generate an automatic navigation instruction for reducing the advancing speed, and an automatic operation is executed at a lower advancing speed, thereby improving the safety of the operation. If the number of the branches of the blood vessel does not exceed the first threshold, the step S403 is continuously executed to determine whether the curvature exceeds the second threshold, and if the curvature exceeds the second threshold, it indicates that the degree of bending of the blood vessel is large, which is likely to cause a safety problem, at this time, the step S405 is also executed to generate an automatic navigation instruction for reducing the advanceing speed, so that the medical interventional device advances on a path with large curvature at a slow speed. In addition, if the curvature does not exceed the second threshold, whether the width of the blood vessel exceeds the third threshold is continuously judged (step S404), and if the width of the blood vessel exceeds the third threshold, the width of the blood vessel is narrow, which is not beneficial to the high-speed advanceing of the medical interventional device, so that an automatic navigation instruction for reducing the advanceing speed is also generated (step S405) to enable the medical interventional device to execute the automatic operation at a safe speed, and the safety of the operation is ensured. The first threshold, the second threshold, and the third threshold may be manually set values or automatically set values by a system, which is not particularly limited. For different blood vessel conditions, different speed modes can be selected, different strategies are given, the operation time is saved, and the operation efficiency is improved. Based on the above analysis and calculation, the optimal path and the optimal execution mode are presented to the physician on the display 1015, and a plurality of alternative execution modes are provided, so that the physician needs to confirm the system to proceed to the next step according to the actual conditions of the patient, such as illness state. The above is only one example and is not intended to limit the scope of protection.

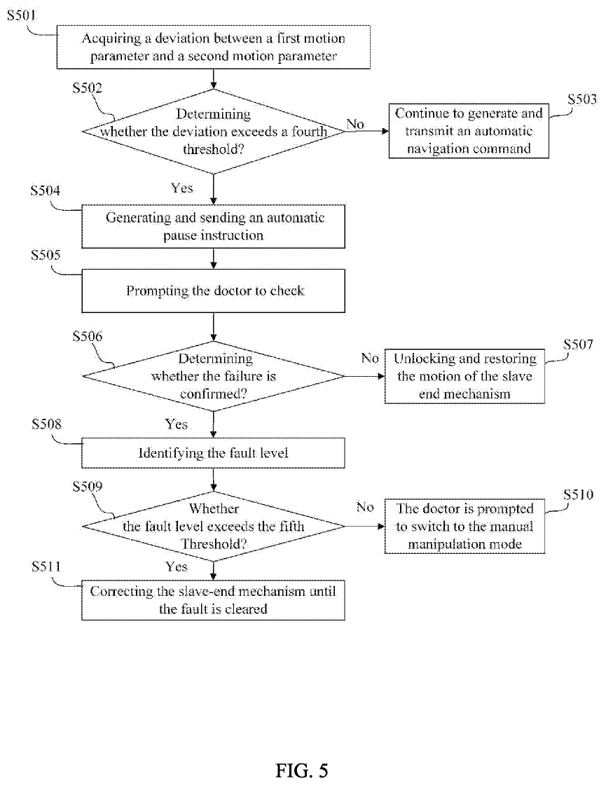

In some embodiments, the at least one processor is further configured to receive a first motion parameter from the slave-end mechanism for manipulating the medical interventional device, determine a second motion parameter of the medical interventional device based on the intra-operative image, compare the first motion parameter and the second motion parameter to determine a deviation, and continue to generate and transmit the automatic navigation instruction in the event that the determined deviation does not exceed a fourth threshold, thereby ensuring safe performance of the automatic procedure. Specifically, as shown in FIG. 5, in step S501, a deviation between a first motion parameter and a second motion parameter is acquired. The first motion parameter reflects a value of a relevant motion parameter expected and set by a doctor for manipulating a medical interventional device, and the second motion parameter reflects a value of a relevant motion parameter actually acquired after manipulating the medical interventional device, and the deviation between the first motion parameter and the second motion parameter can reflect the accuracy of the automatic operation, and the smaller the deviation, the better the execution result of the automatic operation. The deviation may be a difference between the first motion parameter and the second motion parameter, and the deviation may be calculated directly by the system, or may be calculated manually by a doctor or calculated in other feasible manners, which is not limited in particular. In step S502, whether the deviation exceeds a fourth threshold is determined. If not, the step S503 is executed to continue generating and transmitting an automatic navigation command, and an automatic operation is safely and efficiently executed according to the previous setting.

Further, in step S504, in case that the determined deviation exceeds the fourth threshold, an automatic pause instruction is generated and sent, which causes the motion of the slave-end mechanism 102 to pause, and the current state of the slave-end mechanism 102 is maintained and the doctor is prompted to check (step S505). Next, step S506 is executed to determine whether the failure is confirmed, and when the failure is cleared as a result of the check, the slave-end mechanism 102 is unlocked and restored to the motion (step S507), so that the slave-end mechanism 102 continues to operate the medical interventional device to perform the automatic operation. And when the checking result is that the fault is confirmed. identifying the fault level (step S508), and performing corresponding correction according to the fault level. In step S509, it is confirmed whether the fault level does not exceed the fifth threshold, and when the identified fault level is equal to or lower than the fifth threshold, step S511 is executed to correct the slave-end mechanism 102 until the fault is cleared, specifically including continuing to lock and maintain the current state of the slave-end mechanism 102, while automatically or semi-automatically controlling the slave-end mechanism 102 to increase at least one of the clamping force and the propulsive force and prompting the doctor to check until the check result becomes the fault clearing. The clamping force and the propelling force are critical to control the movement of the medical interventional device in the cavity, and when the clamping force or the propelling force cannot meet the requirement, the medical interventional device cannot be accurately controlled, for example, the medical interventional device falls off in the movement process due to the fact that the clamping force is low. Such a malfunction may be addressed by increasing the clamping force, and therefore, when the system indicates a malfunction level at or below a fifth threshold level, the slave-end mechanism 102 of the interventional surgical robot need not be shut down, as long as the clamping or propulsion force is increased by adjustment. Furthermore, other methods by which a fault can be eliminated by adjustment of the system are not excluded.

When the identified fault level is higher than the fifth threshold, the slave-end mechanism 102 is turned off, and the doctor is prompted to switch to the manual manipulation mode (step S510). At this time, a higher fault level means that the system has a serious problem that the system is difficult to repair through simple adjustment, and therefore, when the identified fault level is higher than the fifth threshold level, the slave-end mechanism 102 of the interventional surgical robot is turned off, and then manual operation and control are performed by a doctor, so that damage to a patient caused by system fault is avoided, and the life safety of the patient is ensured. Through the embodiment, a series of problems that the existing interventional surgical robot has no abnormal protection mechanism, the interventional robot cannot judge the abnormal state of the operation, the interventional robot has no real-time monitoring abnormal state, and when abnormal conditions occur, the robot does not stop in time, real-time feedback information does not exist and the like can be solved. In addition, the control cabinet 1014 may prompt the doctor for a check by issuing an alarm in case the determined deviation exceeds the fourth threshold. The fourth threshold and the fifth threshold may be manually set numerical values or default numerical values of the system, which is not limited herein.

In some embodiments, the at least one processor receives the motion resistance data and the motion trail data from the slave mechanism for manipulating the medical interventional device, the display displays the motion resistance data and the motion trail data, and based on the motion resistance data and the motion trail data, a doctor can judge the current motion state of the medical interventional device and can judge whether certain danger exists.

In some embodiments, analyzing the representative image to obtain the planning path particularly includes analyzing the representative image through a learning network to segment the physiological tubular structure. For example, the acquired medical image information, is subjected to image preprocessing and input into a ResUnet deep learning network for training, target objects such as guide wires, stents, blood vessels and the like are identified, training data are acquired, shuffle operation is performed on the data, the images are converted into fixed sizes (such as 512×512), normalization processing is performed, pixels are converted into 0-1, the training data comprise medical images of segmentation marking information (blood vessels, guide wires and stents), image processing methods such as image horizontal turning, vertical turning, random scaling, random brightness, random contrast, random noise and the like are performed on the training data for data enhancement, and the enhanced training data are used for learning and training a segmentation network model to obtain an image segmentation model. Image preprocessing is carried out on the acquired medical image information, the acquired medical, image information is input into a ResUnet deep learning network for training, a training result is compared with training data, a loss value is calculated through a cross entropy loss function calculation method, and the loss value is subjected to reverse propagation to update the weight. The extraction of blood vessels and other features is carried out through a deep learning network, the extraction efficiency of the features can be greatly improved, and the method is a fundamental guarantee for realizing real-time navigation. The deep learning network model may be a segmentation network model such as ResUnet and attentionUnet, and is not particularly limited. The segmentation network model is learned and trained by adopting the training data of the medical images of various segmentation labeling information (blood vessels, guide wires and supports), so that the image segmentation model can be acquired, and the accuracy and the speed of segmenting the segmentation target by using the acquired image segmentation model are ensured. The deep learning network can be realized by utilizing a Tensorflow framework to carry out deep learning training.

In the automatic operation process, the interventional, robot system, transmits a real-time image into a trained network model, the network model outputs a recognition result according to a trained rule, a physiological tubular structure is segmented, and doctor operation data and a network model output result are converted into a navigation instruction through image processing calculation. The centerline of the physiological tubular structure is extracted with the lesion part as a terminal part, the prediction result of the network blood vessel as a “road”, and a representative part of the medical interventional device (such as a guide wire, a catheter, or a stent) as a starting part. The extracted central, line can be directly used as a planning path, manual intervention can be started to correct the central line, and the corrected central line is used as the planning path again to ensure the safety of the robot operation.

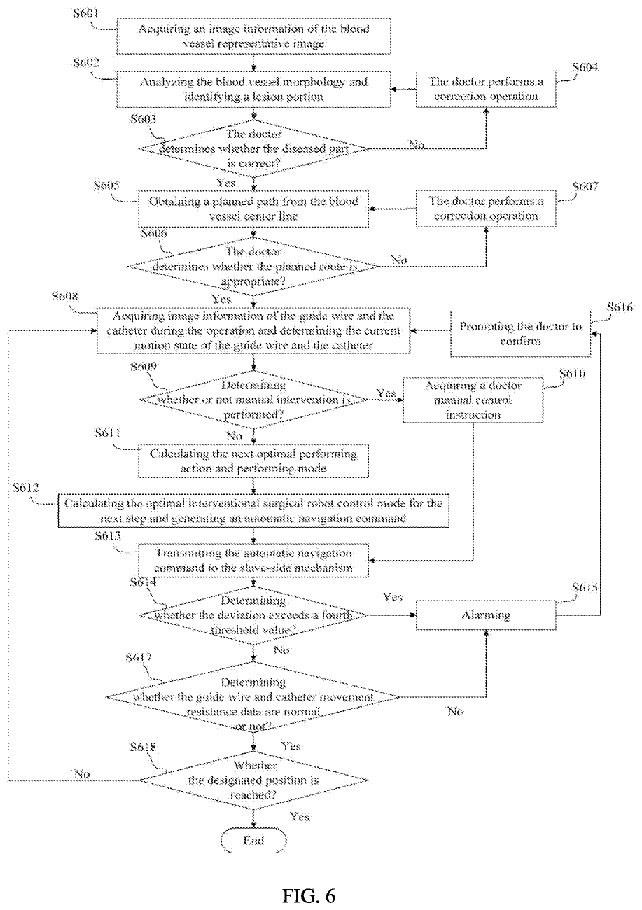

In some embodiments, the at least one processor 1011 is further configured to perform analysis processing on the representative image to identify a lesion. The display 1012 is further configured to display the identified lesion to facilitate the doctor to confirm whether the confirmation of the lesion position is correct. As shown in FIG. 6, in step S601, image information of the blood vessel representative image is acquired and transmitted to the control cabinet 1014, and the control cabinet 1014 analyzes the blood vessel morphology and identifies a lesion portion (step S602). Specifically, after the control cabinet 1014 obtains the blood vessel image of the patient, the system performs automatic matching analysis based on a large amount of data to diagnose the disease condition of the blood vessel and find the diseased part (e.g., a narrow region), at this time, the system pops up a prompt to confirm whether the disease condition analysis is accurate by the doctor, that is, the doctor determines whether the diseased part is correct by performing step S603. The at least one processor 1011 is further configured to receive user interaction with the lesion, the interaction including at least one of confirmation, correction, and rejection. If the doctor judges that the lesion position is correct, the doctor performs the confirmation manipulation and then proceeds to step S605. If the doctor determines that the lesion position is erroneous, the doctor performs a correction operation in step S604. Or, the doctor directly executes rejection, operation, and the system analyzes and processes the image information again. For example, the physician may adjust system analysis metrics and parameters for re-analysis by the system. Alternatively, the contrast image to be manipulated is specified by the doctor, the manipulation command is not limited to the form including, but not limited to, drawing a dot, drawing a circle, drawing a rectangle, drawing a line, etc. on the screen image, and the robot system acquires the lesion as the terminal portion by recognizing the doctor manipulation command. The processor 1011, upon receiving confirmation (possibly subjected to a correction operation) of the lesion by the user, obtains the planning path with the confirmed lesion as a terminal portion.

In step S605, a blood vessel center line is extracted using a learning network based on the identified lesion as a terminal portion, and a planning path is acquired from the blood vessel center line. Further, the display unit 1012 is further configured to display the planned route, so that the physician can determine the accuracy of the planned route. In step S606, the doctor determines whether the planned route is appropriate. For example, the planning path may not match the actual path due to inaccurate extraction of the centerline of the blood vessel, and at this time, the accuracy and the safety of the subsequent automatic surgery are improved by manual intervention. Further, the at least one processor 1011 is further configured to receive user interactions with the planning path, the interactions including at least one of confirmations, revisions, and rejections. Specifically, after receiving a correction operation of the planning path by the user, the planning path is corrected in response to the correction operation and displayed on the display unit 1012. For example, when the doctor determines that the planning path is not appropriate, the doctor performs a correction operation (step S607), and the processor 1011 receives the correction operation of the doctor, corrects the planning path, and displays the corrected appropriate planning path on the display 1015, so that the next step can be performed until the planning path meets the requirements of the doctor. The processor 1011, upon receiving a confirmation operation of the planning path by the user, generates an automatic navigation instruction based on the confirmed planning path and the current motion state of the medical interventional device. After the planning path is acquired, the system calculates how to control the slave-end mechanism 102 to act, and the concrete expression may include calculating the rotation direction, rotation angle, rotation speed, etc. of each motor, and these control information are used as navigation instructions, sending the navigation instructions to the slave-end mechanism 102 of the robot, and starting to move, and simultaneously acquiring the image information of the DSA104 in real time, and the system automatically judges the position information of the guide wire and the catheter. Specifically, as shown in step S608, the processor 1011 acquires image information of the guide wire and the catheter during the operation and determines the current motion state of the guide wire and the catheter, such as determining the motion parameters related to the motion direction, the position, and the like of the guide wire and the catheter. Next, the system determines whether or not manual intervention is performed (step S609), and if manual intervention is required, the processor 1011 acquires a doctor manual control instruction (step S610) and transmits an automatic navigation instruction to the slave terminal mechanism 102 (step S613). For example, when an abnormal condition occurs during the operation, the doctor can suspend the automatic operation system at any time and adjust the automatic operation system in a manual operation mode. When the manual adjustment is completed, the automatic surgical mode may be returned again. If no manual intervention is needed, the step S611 is executed to calculate the next optimal performing action and performing mode, for example, the next optimal performing action and performing mode are calculated by the method of confirming the for and automatic navigation command, the forward distance automatic navigation command, and the rotational automatic navigation command described in the above embodiments, and are converted into the action command that can be performed by the machine, and the action information is displayed on the display 1015 to inform the doctor. Next, in step S612, the optimal interventional surgical robot control mode for the next step is calculated, an automatic navigation command is generated, the automatic navigation command is transmitted to the slave side mechanism 102 (step S613), and the slave side mechanism 102 executes the automatic surgery based on the automatic navigation command. The doctor evaluates the operation result of the interventional surgical robot, calculates the deviation of the first motion parameter of the guide wire and the catheter operated by the slave-end mechanism 102 and the second motion parameter associated with the actual result through the system, judges whether the deviation exceeds a fourth threshold value (step S614), alarms by the system (step S615) if the deviation exceeds the fourth threshold value, prompts the doctor to confirm (step S616), and checks and corrects the operation process by the doctor. If the deviation does not exceed the fourth threshold, judging whether the guide wire and catheter movement resistance data are normal or not (step S617), if not, alarming by the system, prompting a doctor to check and correct, and if so, continuing to perform the automatic operation. The display 1015 displays the intra-operative image, and the physician can observe whether the guide wire and the catheter reach the designated position at any time (step S618), and if the guide wire and the catheter reach the designated position, the process ends, and if the guide wire and the catheter do not reach the designated position, the process returns to step S608 to continue the process. Therefore, the execution efficiency of the automatic operation can be improved, the accuracy and the safety of the automatic operation are greatly improved through human-computer interaction, and the efficiency and the success rate of the automatic operation can be greatly improved.

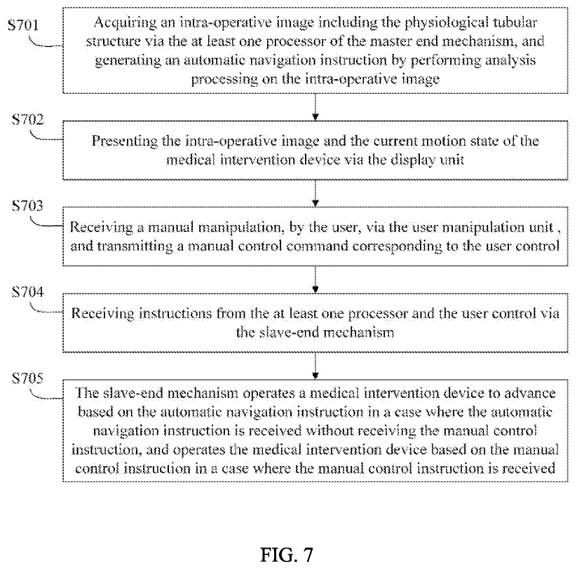

FIG. 7 shows a flowchart of a control method of an interventional surgical robot according to an embodiment of the disclosure. Wherein the interventional surgical robotic system 10 is used to manipulate a medical interventional device for movement within a lumen of a physiologic tubular structure of a patient. In step S701, an intra-operative image including the physiological tubular structure is acquired via the at least one processor 1011 of the master-end mechanism 101, and an automatic navigation instruction is generated by performing analysis processing on the intra-operative image. In step S702, the intra-operative image and the current motion state of the medical interventional device are presented via the display unit 1012. In step S703, a manual manipulation by the user is received via the user control 1013, and a manual control command corresponding to the manual manipulation is transmitted. In step S704, instructions from the at least one processor 1011 and the user control 1013 are received via the slave-end mechanism 102, wherein the slave-end mechanism 102 is provided with a robot arm 1021 and an end actuator 1022. In step S705, the slave-end mechanism 102 operates a medical interventional device to advance based on the automatic navigation instruction in a case where the automatic navigation instruction is received without receiving the manual control instruction, and operates the medical interventional device based on the manual control instruction in a case where the manual control instruction is received. Therefore, the control method of the interventional surgical robot with manual monitoring and manual adjustment is provided, and meanwhile, various surgical parameters are provided for doctors, and the current operation and the next operation are performed, so that the doctors can know the state of the robot conveniently. The doctor can stop and correct at any time, and can continue to perform the operation automatically after the correction is completed.

In some embodiments, acquiring an intra-operative image including the physiological tubular structure, generating an automatic navigation instruction by analyzing the intra-operative image specifically includes acquiring a representative image including the physiological tubular structure, analyzing the representative image to obtain a planning path, analyzing the intra-operative image to determine a current motion state of the medical interventional device, and generating the automatic navigation instruction based on, the planning path and the current motion state of the medical interventional device. Therefore, automatic interventional operation can be completed, optimal path planning is adopted, more efficient image analysis and robot execution strategies are achieved, and the efficiency and the success rate of automatic operation can be greatly improved.

In some embodiments, the generating of the automatic navigation instruction based on the planning path and the current motion state of the medical interventional device specifically includes acquiring a current first position and a first motion direction of a representative portion of the medical interventional device, determining a second position of the representative portion in the first motion direction, determining a shortest connecting line between the second position of the representative portion and the planning path, determining an intersection point of the shortest connecting line and the planning path, acquiring an included angle between a connecting line of the intersection point and the first position and the first motion direction, and when the included angle is smaller than a first threshold angle, generating an advancing automatic navigation instruction to advance the medical interventional device along the planning path, so as to ensure safety.

In some embodiments, generating the automatic navigation instruction based on the planning path and the current motion state of the medical interventional device further comprises determining a distance between the second location and the first location as an advancing distance when the included angle does not exceed a first threshold angle.

In some embodiments, generating an automatic navigation instruction based on the planning path and the current motion state of the medical interventional device further comprises determining an, angle by which the medical interventional device is to be steered to rotate when the included angle is greater than a first threshold angle, and generating an automatic rotation instruction indicating the angle of rotation as the automatic navigation instruction.

Therefore, the control method based on the interventional surgical robot can reduce the physical burden of a doctor and improve the operation experience of the doctor, and is simple and easy to implement.

Embodiments of the disclosure also provide a computer-readable storage medium having stored thereon computer program instructions, which, when executed by the processor 1011, cause the processor 1011 to perform a method of controlling an interventional surgical robot according to various embodiments of the disclosure. The storage medium may include read-only memory (ROM), flash memory, random-access memory (RAM), dynamic random-access memory (DRAM) such as Synchronous DRAM (SDRAM) or Rambus DRAM, static memory (e.g., flash memory, static random-access memory), etc., on which computer-executable instructions may be stored in any format.

Moreover, although exemplary embodiments have been described herein, the scope thereof includes any and all embodiments based on the disclosure with equivalent elements, modifications, omissions, combinations (e.g., of various embodiments across), adaptations or alterations. The elements of the claims are to be interpreted broadly based on the language employed in the claims and not limited to examples described in the present specification or during the prosecution of the disclosure, which examples are to be construed as non-exclusive. It is intended, therefore, that the specification and examples be considered as exemplary only, with a true scope and spirit being indicated by the following claims and their full scope of equivalents.

The above description is intended to be illustrative, and not restrictive. For example, the above-described examples (or one or more versions thereof) may be used in combination with each other. For example, other embodiments may be utilized by those of ordinary skill in the art upon reading the foregoing description. In addition, in the above detailed description, various features may be grouped together to streamline the disclosure. This should not be interpreted as an intention that a non-claimed disclosed feature, is essential to any claim. Rather, subject matter of the disclosure may lie in less than all features of a particular disclosed embodiment. Thus, the following claims are hereby incorporated into the detailed description as examples or embodiments, with each claim standing on its own as a separate embodiment, and it is contemplated that the, embodiments can be combined with each other in various combinations or permutations. The scope of the invention should be determined with reference to the appended claims, along with the full scope of equivalents to which such claims are entitled.

The above embodiments are only exemplary embodiments of the disclosure, and are not intended to limit the present invention, the scope of which is defined by the claims. Various modifications and equivalents may be made by those skilled in the art within the spirit and scope of the disclosure and such modifications and equivalents should also be considered as falling within the scope of the present invention.

What is claimed is:

1. An interventional surgical robotic system for manipulating a medical interventional device to advance within a lumen of a physiological tubular structure of a patient, comprising: a master-end mechanism, comprising:
at least one processor configured to:
acquire an intra-operative image containing the physiological tubular structure, and generate an automatic navigation instruction by performing an analysis on the intra-operative image;
a display for displaying the intra-operative image and a current motion state of the medical interventional device; and
a user control configured to:
receive a manual operation of a user and transmitting a manual control instruction corresponding to the manual operation;
a slave-end mechanism provided with a robot arm and an end actuator, and configured to:
receive instructions from the at least one processor and the user control;
manipulate the medical interventional device to advance based on the automatic navigation instruction in the event that an automatic navigation instruction is received without receiving the manual control instruction, or manipulate a medical, interventional device based on the manual control instruction in the event that the manual control instruction is received.

2. The interventional surgical robotic system of claim 1, wherein the step of acquiring an intra-operative image containing the physiological tubular structure, and generating an automatic navigation instruction by performing an analysis on the intra-operative image comprises
acquiring a representative image containing a physiological tubular structure, and analyzing and processing the representative image to obtain a planning path;
analyzing the intra-operative image to determine a current motion state of a medical interventional device; and
generating an automatic navigation instruction based on the planning path and a current motion state of the medical interventional device.

3. The interventional surgical robotic system of claim 2, wherein generating automatic navigation instructions based on the planning path and the current motion state of the medical interventional device specifically comprises:
obtaining a current first position and a first direction of motion of a representative portion of the medical interventional device;
determining a second position of the representative portion in the first direction of motion;
determining a shortest connecting line between the second position of the representative pant and the planning path;
determining an intersection point of the shortest connecting line and the planning path;
acquiring an included angle between a connecting line of the intersection point and the first position and the first movement direction; and
generating an advancing automatic navigation instruction when the included angle is smaller than a first threshold angle.

4. The interventional surgical robotic system of claim 3, wherein the step of generating automatic navigation instructions based on the planning path and a current motion state of the medical interventional device further comprises:
determining a distance between the second position and the first position as an advancing distance when the included angle does, not exceed a first threshold angle.

5. The interventional surgical robotic system of claim 3, wherein the step of generating an automatic navigation instruction based on the planning path and a current motion state of the medical interventional device further comprises:
determining an angle at which the medical intervention device is to be manipulated to rotate when the included angle is greater than a first threshold angle, and generating an automatic rotation instruction instructing to rotate the angle as the automatic navigation instruction.

6. The interventional surgical robotic system of claim 2, wherein the planning path remains stable during an operation, and the image of the physiological tubular structure includes a vessel image of at least one of a neural vessel, a visceral vessel, and a peripheral vessel.

7. The interventional surgical robotic system of claim 1, wherein the manual control instructions include at least one of an automatic pause instruction, an automatic resume instruction, a planning path revision instruction, and a manual navigation instruction, the display being further configured to display a planning path, the displayed planning path being manually altered in response to the planning path revision instruction.

8. The interventional surgical robotic system of claim 1, wherein the at least one processor is further configured to:
analyzing the intra-operative image to determine vessel branching and bending conditions and vessel width prior to advancing of the medical interventional device; and
generating an automatic navigation instruction for reducing the advance speed of the medical interventional device if the number of blood vessel branches ahead exceeds a first threshold value, or the curvature is greater than a second threshold value, or the width of the blood vessel is less than a third threshold value.

9. The interventional surgical robotic system of claim 1, wherein the at least one processor is further configured to:
receive a first motion parameter from the slave-end mechanism for manipulating the medical interventional device;
determine a second motion parameter of the medical interventional device based on the intra-operative image;
compare the first motion parameter and the second motion parameter to determine a deviation; and
continuously generating and sending an automatic navigation instruction under the condition that the determined deviation does not exceed the fourth threshold value.

10. The interventional surgical robotic system of claim 9, wherein the at least one processor is further configured to:
generate and send an automatic pause instruction under the condition that the determined deviation exceeds a fourth threshold value; wherein the automatic pause instruction enables the motion of the slave-end mechanism to pause, and the current state of the slave-end mechanism is maintained in a locking mode and prompts a doctor to check;
unlock and recover the motion of the slave-end mechanism when the checking result is that the fault is cleared;
identify the fault level when the checking result is that the fault is confirmed;
continuing to lock and maintain the current state of the slave-end mechanism, and simultaneously automatically or semi-automatically control the slave-end mechanism to increase at least one of clamping force and propelling force and prompte a doctor to check until the check result becomes fault-free when the identified fault, level is equal to or lower than a fifth threshold value; and
enable the, slave-end mechanism and prompte the doctor to switch to a manual operation mode when the identified fault level is higher than the fifth threshold value.

11. The interventional surgical robotic system of claim 9, wherein an alarm is issued in case the determined deviation exceeds a fourth threshold value.

12. The interventional surgical robotic system of claim 1, wherein the at least one processor receives motion resistance data and motion trajectory data from the slave-end mechanism for manipulating the medical interventional device, the motion resistance data and motion trajectory data being displayed by a display.

13. The interventional surgical robotic system of claim 2, wherein analyzing the representative image to obtain a planning path specifically comprises:
analyzing and processing the representative image through a learning network to segment the physiological tubular structure;
taking a representative part of the medical interventional device as an initial part and a lesion part as a terminal part, and extracting a central line of the physiological tubular structure; and
obtaining the planning path according to the extracted central line.

14. The interventional surgical robotic system of claim 2, wherein the at least one processor is further configured to:
analyzing the representative image to identify a lesion;
the display is further configured to display the identified lesion;
the at least one processor, is further configured to:
receiving interactive operation of the lesion part by a user, wherein the interactive operation comprises at least one of confirmation, correction and rejection;
upon receiving confirmation of the lesion by the user, the planning path is acquired with the confirmed lesion as a terminal portion.

15. The interventional surgical robotic system of claim 7, wherein the display is further configured to display the planning path; the at least one processor is further configured to:
receive interactive operation of a user on the planning path, wherein the interactive operation comprises at least one of confirmation, correction and rejection; generate the automatic navigation instruction based on the confirmed planning path and the current motion state of the medical interventional device after receiving a confirmation operation of the planning path by a user; and
correct the planning path in response to the correcting operation for display by the display after receiving the correcting operation of the user on the planning path.

16. A method for controlling an interventional surgical robot for manipulating a medical interventional device to move in a lumen of a physiological tubular structure of a patient, comprising:
acquiring an intra-operative image containing the physiological tubular structure via at least one processor of a master-end mechanism, and generating an automatic navigation instruction by performing an analysis on the intra-operative image;
displaying the intraoperative image and a current motion state of the medical interventional device via a display;
receiving a manual operation of a user, via a user control and transmitting a manual control instruction corresponding to the manual operation;
receiving instructions from the at least one processor and the user control via a slave-end mechanism; wherein the slave-end mechanism is provided with a robot arm and an end actuator, and configured to:
manipulate the medical interventional device to advance based on the automatic navigation instruction in the event that an automatic navigation instruction is received without receiving the manual control instruction, or manipulate a medical interventional device based on the manual control instruction in the event that the manual control instruction is received.

17. The method of claim 16, wherein the step of acquiring an intra-operative image containing the physiological tubular structure, and generating an automatic navigation instruction by performing an analysis on the intra-operative image comprises:
acquiring a representative image containing a physiological tubular structure, and analyzing and processing the representative image to obtain a planning path;
analyzing the intra-operative image to determine a current motion state of a medical interventional device; and
generating an automatic navigation instruction based on the planning path and a current, motion state of the medical interventional device.

18. The method of claim 17, wherein the step of generating automatic navigation instructions based on the planning path and the current motion state of the medical interventional device specifically comprises:
obtaining a current first position and a first direction of motion of a representative portion of the medical interventional device;
determining a second position of the representative portion in the first direction of motion;
determining a shortest connecting line between the second position of the representative part and the planning path;
determining an intersection point of the shortest connecting line and the planning path;
acquiring an included angle between a connecting line of the intersection point and the first position and the first movement direction; and
generating an advancing automatic navigation instruction when the included angle is smaller than a first threshold angle.

19. The method of claim 18, wherein the step of generating automatic navigation instructions based on the planning path and a current motion state of the medical interventional device further comprises:
determining a distance between the second position and the first position as an advancing distance when the included angle does not exceed a first threshold angle.

20. The method of claim 18, wherein the step of generating automatic navigation instructions based on the planning path and a current motion state of the medical interventional device further comprises:
determining an angle by which the medical interventional device is to be manipulated to rotate and generating an automatic rotation instruction indicating the rotation of the angle as the automatic navigation instruction when the included angle is greater than a first threshold angle.
